(19) United States
(12) Patent Application Publication
(10) Pub. No.: US 2021/0187225 A1
(43) Pub. Date: Jun. 24, 2021

(54) INTUBATION ASSIST DEVICE

(71) Applicant: ELICORE MEDICAL SOLUTIONS, LLC, Johnston, RI (US)

(72) Inventors: Vito Ferri, Cranston, RI (US); Christopher Ferri, Cranston, RI (US); Richard Pedro, Assonet, MA (US); Louis Insalaco, Wilmington, MA (US); Ryan Murphy, West Orange, NJ (US); Cary Vachon, Schenectady, NY (US)

(21) Appl. No.: 16/725,328

(22) Filed: Dec. 23, 2019

(51) Int. Cl. A61M 16/04

(52) U.S. Cl. CPC A61M 16/0463; A61M 16/0488; A61M 16/0484

(57) ABSTRACT

Embodiments of the present invention disclose a suction stylet assembly for endotracheal intubation. The assembly includes an elongated tubular member, the elongated tubular member having one or more apertures proximate to a first end, and a handle in communication with a second end of the elongated tubular member. The handle member includes a first passageway connecting the elongated tubular member to a hose fitting on the handle member, the hose fitting configured to receive a hose that provides suction in removing bodily fluids. The handle member further includes an aperture located on a side of the handle member, the aperture forming a second passageway through the handle member running between the side and the first passageway, the aperture configured to permit a user to selectively block the aperture, thereby selectively controlling suction provided between the hose fitting and the elongated tubular member.

### BACKGROUND

### 1. Field of the Invention

The present application relates to endotracheal tubes, and more particularly to stylets that guide endotracheal tubes for intubation.

### 2. Description of Related Art

A common procedure performed by medical professionals to assist a patient to breathe is intubation through the use endotracheal tubes. This may be performed during surgery or during prolonged need for breathing assistance, such as a stay in an intensive care unit. A standard endotracheal tube has a cuff located proximately at a first end of the tube to seal the end of the tube against the inner walls of a trachea where a pilot balloon connects to the cuff to inflate the cuff. During a standard intubation, the medical professional will commonly place a malleable stylet inside the endotracheal tube and then bend the endotracheal tube and stylet together to a desired shape to optimize intubation. Once the endotracheal tube is in the desired location in the trachea, the stylet is removed, and the endotracheal tube is left in place in the trachea. The cuff is then inflated, and a ventilator circuit is attached to a connector located at a second end of the tube.

During intubation, the medical professional that is intubating the patient uses one hand to expose an opening to the patient's windpipe while the professional's second hand maneuvers the endotracheal tube. Often during this process, the medical professional must use their second hand to suction mucous, blood, or vomit from the patient's throat in order to obtain an adequate view of the vocal cords, between which the endotracheal tube is placed. However, to remove these fluids, the professional must set aside the endotracheal tube to use a suction device, which can waste valuable time in often crucial periods where the patient's airway is not secured. Therefore, it is desired to develop a device that provides for simultaneous suction and intubation without having to use a separate suction device.

### SUMMARY OF THE INVENTION

Embodiments of the present invention disclose a suction stylet assembly for endotracheal intubation. In one embodiment of the present invention, an assembly is provided comprising: an elongated tubular member having a first end and a second end, the elongated tubular member having one or more apertures proximate to the first end; a handle member having a first end, a second end, and a side that runs between the first end and the second end, the first end of the handle member in communication with the second end of the elongated tubular member, the handle member including: a first passageway passing through the handle member running from the first end to the second end, the elongated tubular member coaxial with the first passageway; a hose fitting located at the second end, the hose fitting configured to receive a hose that provides suction in removing bodily fluids; and an aperture located on the side, the aperture forming a second passageway through the handle member running between the side and the first passageway, the aperture configured to permit a user to selectively block the aperture with a digit of the user, thereby selectively controlling suction provided between the hose fitting of the handle member and the first end of the elongated tubular member.

Ultimately the invention may take many embodiments. In these ways, the present invention overcomes the disadvantages inherent in the prior art.

The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the various purposes of the present design. It is important, therefore, that the claims be regarded as including such equivalent constructions in so far as they do not depart from the spirit and scope of the present application.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a side view of a stylet assembly having a sheath portion for coupling with a connector of an endotracheal tube, in accordance with an embodiment of the present invention;

FIG. 2 is a top view of the stylet assembly of FIG. 1;

FIG. 3 is a side view of an endotracheal tube having a connector;

FIG. 4 is a top view of the endotracheal tube of FIG. 3;

FIG. 5 is a side view of the stylet assembly of FIG. 1 coupled with the endotracheal tube of FIG. 3;

FIG. 6 is a top view of the stylet assembly and endotracheal tube of FIG. 5;

FIG. 7 is a side view of a stylet assembly having a tapered portion for coupling with a connector of an endotracheal tube;

FIG. 8 is a side view of the style assembly of FIG. 7 coupled with the endotracheal tube of FIG. 3; and

FIG. 9 is a view of the suction stylet assembly in position in a patient.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The assembly and method in accordance with the present invention overcomes one or more of the above-discussed problems associated with endotracheal tubes during patient intubation when patient bodily fluids (e.g., mucous, blood, vomit, etc.) occlude breathing passageways to the patient's trachea. In particular, the system of the present invention is a suction stylet having a malleable elongated tubular member and a handle member. The handle member is located at one end of the tubular member and has a first passageway running through the handle member that connects from the tubular member to a hose fitting on the handle member. Furthermore, the handle member has a handle aperture located on a side of the handle member, the aperture being an opening to a second passageway that connects between the side and the first passageway. The handle aperture and associated second passageway permits selective control of suction between a hose fitting (connected to a suction source) and a second end of the tubular member: when the handle aperture is in an open state, suction provided to the hose fitting draws ambient air through the second passageway to the first passageway, thereby redirecting suction away from the tubular member. A user operating the suction stylet assembly directs suction to the tubular member to remove unwanted bodily fluids during intubation by blocking the handle aperture, thereby directing suction from the hose fitting to the tubular member.

The elongated tubular member of the suction stylet is configured to slide into an endotracheal tube through a connector located at one end of the endotracheal tube. The handle member of the suction stylet serves as a stop against the connector to restrict the suction stylet from further insertion into the endotracheal tube. The assembly may come in various sizes to correspond to various endotracheal tube sizes. Furthermore, the elongated tubular member may have a murphy eye that concentrically aligns with a murphy eye typically found on endotracheal tubes. Through the combination of the features listed above, the suction stylet thus allows for simultaneous suction and intubation.

The system will be understood from the accompanying drawings, taken in conjunction with the accompanying description. Several embodiments of the system may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The system of the present application is illustrated in the associated drawings. As used herein, “system” and “assembly” are used interchangeably. As used herein, a “fastener” is a rod-like hardware device that mechanically joins or affixes two or more members together through a respective concentric set of apertures. For example, a fastener can be a screw, bolt, nail, stud, dowel, rivet, staple, etc. in conjunction with any applicable nuts and washers generally known in the art of fastening. It should be noted that the articles “a”, “an”, and “the”, as used in this specification, include plural referents unless the content clearly dictates otherwise. Additional features and functions are illustrated and discussed below.

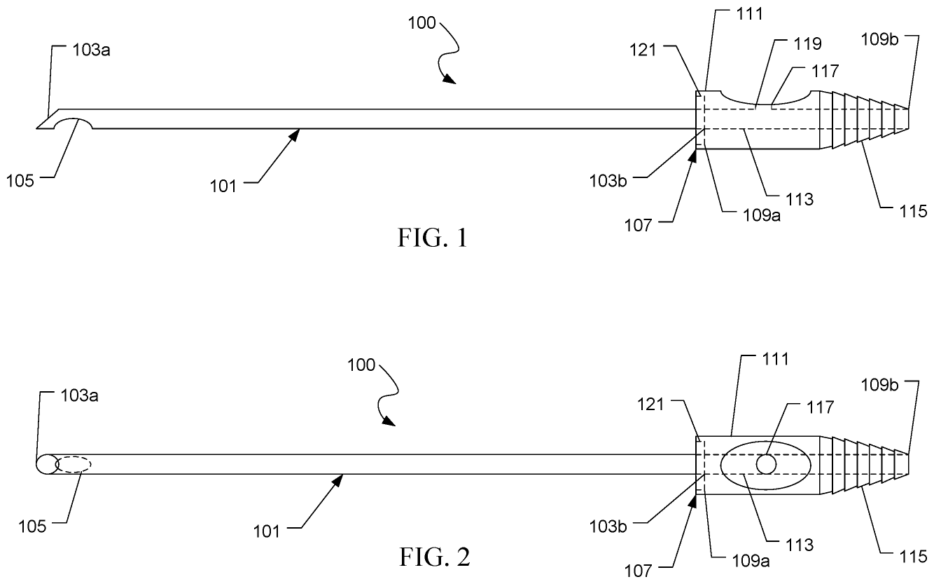

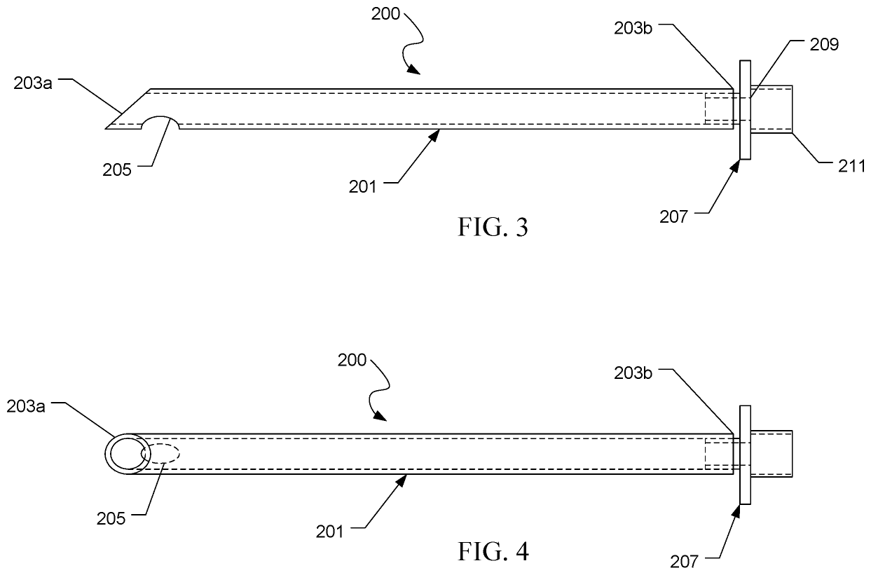

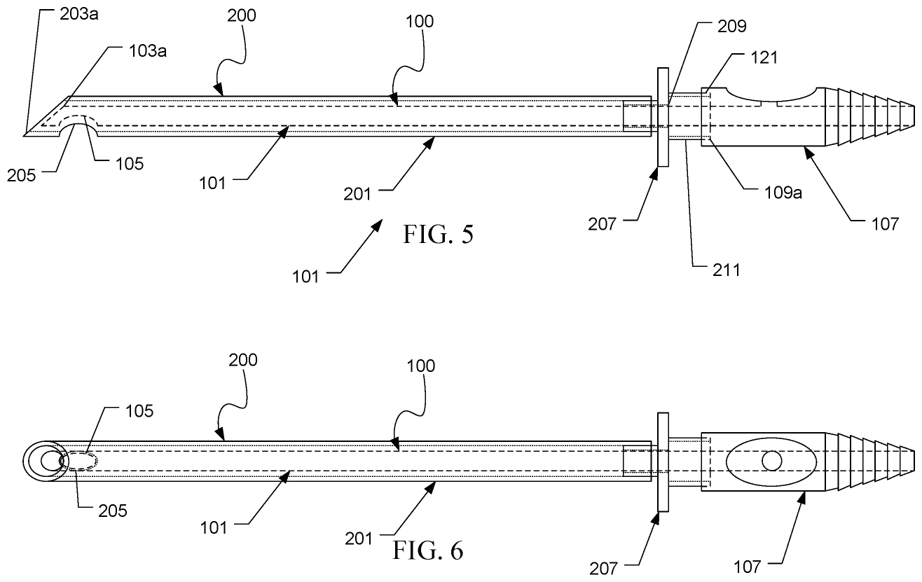

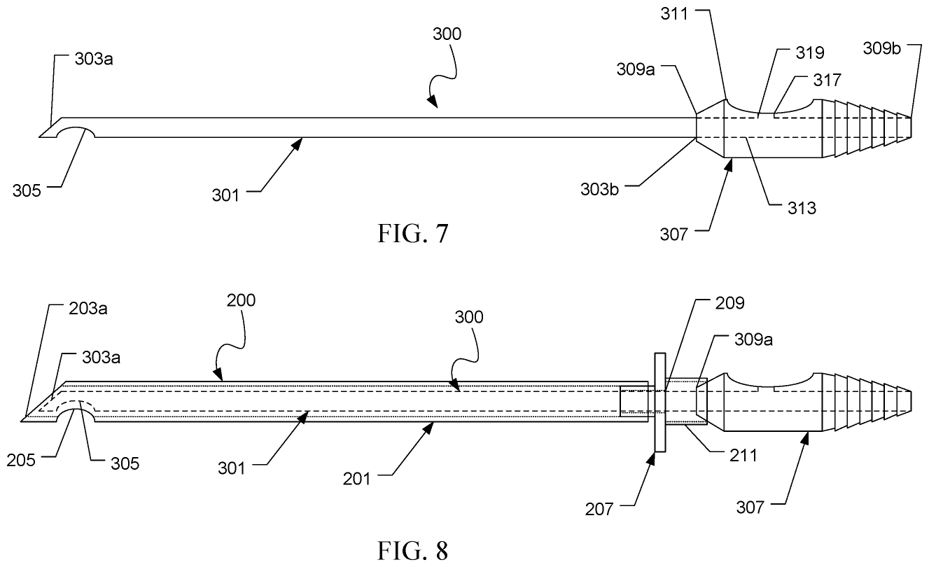

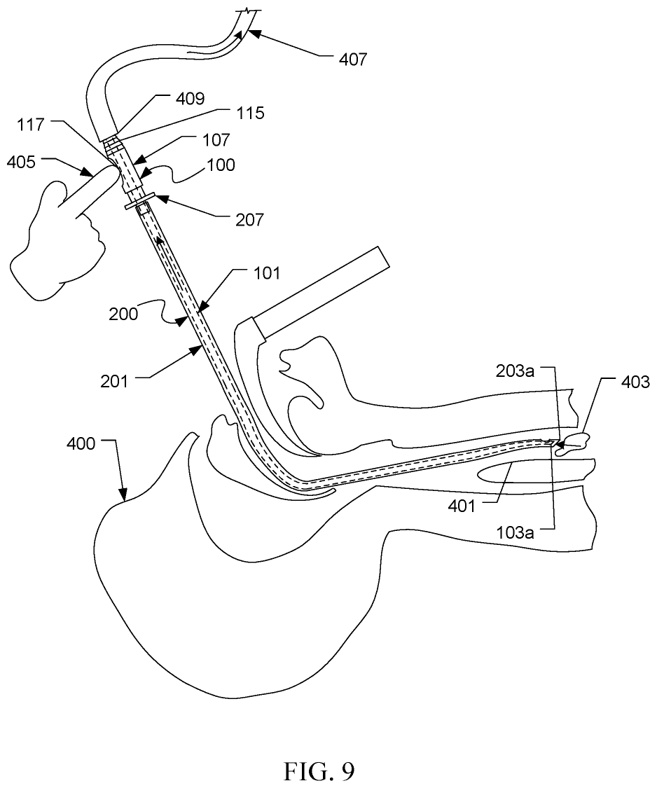

Referring now to the drawings wherein like reference characters identify corresponding or similar elements in form and function throughout the several views. FIGS. 1-2 illustrate various views of a stylet assembly having a sheath portion for coupling with a connector of an endotracheal tube. FIGS. 3-4 illustrate various views of an endotracheal tube generally known in the art. FIGS. 5-6 illustrate various views of the stylet assembly coupled with the endotracheal tube. FIGS. 7-8 illustrate various views of a stylet assembly having a tapered portion for coupling with a connector of an endotracheal tube. FIG. 9 is a view of the suction stylet assembly in position in a patient.

Referring now to FIG. 1, a top view of stylet assembly 100 is illustrated in accordance with an embodiment of the present invention.

In FIG. 1, stylet assembly 100 includes, but is not limited to, elongated tubular member 101 and handle member 107. In general stylet assembly 100 is a suction stylet that serves as a stylet in providing structural support for an endotracheal tube during intubation of a patient as well as providing a suction device for removing unwanted bodily fluids that occlude breathing passages of the patient.

Elongated tubular member 101 is a malleable metal tube. In general, elongated tubular member 101 may be bent by a user into a configuration suitable for patient intubation, wherein once the tubular member 101 is bent into the configuration, tubular member 101 provides structural support for an endotracheal tube during intubation.

Optionally, a flexible plastic rod may be inserted into tubular member 101. The flexible plastic rod and elongated tubular member 101 may be bent together into a configuration suitable for patient intubation, wherein the flexible plastic rod prevents tubular member 101 from kinking while the flexible plastic rod and tubular member 101 are bent into the configuration.

Furthermore, tubular member 101 serves as a conduit for removing unwanted bodily fluids located proximate to a first end to a second end via air suction. Optionally, the elongated tubular member 101 may be coated with a soft synthetic material to allow for low friction removal from an endotracheal tube once patient intubation is successful. Tubular member 101 has ends 103a and 103b having corresponding openings at each end. As used herein, end 103a is a distal end of stylet assembly 100. In some instances, end 103a is a beveled end. Optionally, tubular member 101 has one or more apertures proximate to end 103a. For example, in FIG. 1, aperture 105 is located proximate to end 103a, wherein aperture 105 is a murphy eye similar in form and function as murphy eyes commonly found in the field of endotracheal tubes. However, it should be understood that the one or more apertures, such as aperture 105, may have variable sizes or may be omitted entirely.

Handle member 107 is a handle having ends 109a and 109b. In general, handle member 107 is gripped by a user during intubation, during removal of stylet assembly 100 from an endotracheal tube, and/or during suction of unwanted bodily fluids of a patient; the user being able to selectively control suction at end 103a by selectively blocking an aperture located on the handle associated with a passageway that diverts suction from end 103a to ambient air located externally from the patient. Handle member 107 has side 111 that runs between ends 109a and 109b. End 109a of handle member 107 is in communication with end 103b of tubular member 101. In some instances, handle member 107 is plastic.

Handle member 107 includes, but is not limited to, a first passageway (i.e., passageway 113), a hose fitting (i.e., hose fitting 115), and a side aperture (i.e., aperture 117) associated with a second passageway (i.e., passageway 119). In this figure, passageway 113 passes through handle member 107 running from ends 109a and 109b with tubular member 101 being coaxial with passageway 113. Hose fitting 115 is located at end 109b, wherein hose fitting 115 is a hose fitting configured to receive a hose that provides suction in removing bodily fluids. For example, hose fitting 115 can be a Christmas tree hose fitting as illustrated in FIG. 1. However, it should be understood that a variety of alternate hose fittings may be employed for receiving suction hosing at end 109b.

In this figure, side aperture 117 is located on side 111. Side aperture 117 forms passageway 119 through handle member 107 that runs between side 111 and passageway 113. In this figure, side aperture 117 is configured to permit a user to selectively block aperture 117 with a digit of the user, thereby selectively controlling suction provided between hose fitting 115 and end 103a of tubular member 101.

Furthermore, end 109a of handle member 107 serves as a stop against a connector of an endotracheal tube to restrict stylet assembly 100 further insertion into the endotracheal tube. End 109a may come in a variety of configurations to form a stop. For example, end 109a may have a portion formed as a female connection for receiving the connector of an endotracheal tube as a male connector, or end 109a may have a tapered portion that forms a male connector configured to receive the connector of the endotracheal tube as a female connector. In FIG. 1, handle member 107 has end 109a recessed into handle member 107 to form a sheath portion (i.e., sheath 121) as a female connector that is configured to receive a connector of an endotracheal tube as a male connector. End 109a formed as a male connector is further illustrated and described with respect to FIGS. 7 and 8 (see end 309a).

Referring now to FIG. 2, a top view of stylet assembly 100 of FIG. 1 is illustrated.

Referring now to FIGS. 3 and 4, a side view and top view of endotracheal tube assembly 200 having a connector are illustrated respectively.

In these figures, endotracheal tube assembly 200 is a typical endotracheal tube known in the art having a connector located at one end of the tube. While some endotracheal tubes include a balloon cuff located proximate to an end of the tube that is inserted into a trachea of a patient, the balloon cuff feature has been omitted from the drawings for simplicity. In these figures, endotracheal tube 201 has ends 203a and 203b, wherein end 203a has a beveled end, a murphy eye (i.e., murphy eye 205) is located proximate to end 203a, and connector 207 is in communication with end 203b. In general, connector 207 is a universal or standard endotracheal tube connector that allows for connection to a ventilator circuit once the endotracheal tube is inserted into a trachea of patient. In these figures, connector 207 has aperture 209 and cuff 211, wherein aperture 209 permits air to pass through connector 207, and cuff 211 is a portion of connector 207 that connects to a ventilator circuit.

Referring now to FIGS. 5 and 6, a side view and a top view of stylet assembly 100 coupled with endotracheal tube assembly 200 are illustrated respectively.

In these figures, stylet assembly 100 is coupled with endotracheal tube assembly 200 by inserting end 103a of stylet assembly 100 through aperture 209 of endotracheal tube assembly 200 until connector 211 is in contact with end 109a of handle member 107. In this figure, sheath 121 sheaths an end of connector 211. In this configuration, tubular member 101 is sheathed by endotracheal tube assembly 200. Furthermore, a user aligns stylet assembly 100 with endotracheal tube assembly 200 such that murphy eye 105 of stylet assembly 100 is concentric with murphy eye 205 of endotracheal tube assembly 200.

Referring now to FIG. 7, a side view of stylet assembly 300 having a tapered portion for coupling with connector 207 of endotracheal tube assembly 200 is illustrated.

In this figure, stylet assembly 300 has the same form and function as stylet assembly 100 except for end 309a being a tapered portion of the handle member instead of having sheath 121. In other words, the following components have similar form and function: elongated tubular member 101 is similar to elongated tubular member 301; end 103a is similar to end 303a; end 103b is similar to end 303b; murphy eye similar to murphy eye 305; handle member 107 is similar to handle member 307; end 109b similar to end 309b; side 111 is similar to side 311; passageway 113 is similar to passageway 313; aperture 117 is similar to aperture 317; and passageway 119 is similar to passageway 319. In this figure, end 309a is a tapered portion that forms a male connector configured to receive a connector of an endotracheal tube as a female connector.

Referring now to FIG. 8, a side view of stylet assembly 300 coupled with endotracheal tube assembly 200 is illustrated. In this figure, stylet assembly 300 is coupled with endotracheal tube assembly 200 by inserting end 303a of stylet assembly 300 through aperture 209 of endotracheal tube assembly 200 until connector 211 is in contact with tapered end 309a of handle member 307.

Referring now to FIG. 9, a view of stylet assembly 100 and endotracheal tube assembly 200 in position in a patient. In this figure, a medical professional inserts elongated tubular member 101 of stylet assembly 100 into endotracheal tube assembly 200 through connector 207. The professional then positions handle member 107 in contact with connector 207 and connects end 409 of suction tubing 407 to hose fitting 115, wherein suction tubing 407 provides suction for removing unwanted bodily fluids. The professional then bends tubular member 101 and endotracheal tube 201 into a configuration optimal for intubation, wherein tubular member 101 structurally maintains the bent configuration for endotracheal tube 201. The professional then inserts end 103a of stylet assembly 100 and end 203a of endotracheal tube assembly 200 into trachea 401 of patient 400. To remove fluids 403 that is occluding trachea 401, the professional selectively blocks side aperture 117 on handle member 107 to selectively provide suction to end 103a of tubular member 101 provided by the suction tubing 407, wherein selectively blocking side aperture 117 is performed with digit 405 of the professional. Blocking side aperture 117 provides suction that draws fluids 403 located proximate to end 103a through tubular member 101 and handle member 107, and to connected suction tubing 407. Not blocking side aperture 117 results in ambient air being drawn through aperture 117 and to connected suction tubing 407, thereby diverting suction away from end 103a. Once endotracheal tube assembly 200 is in position in trachea 401 and fluids 403 are removed, the professional removes stylet assembly 100 from endotracheal tube assembly 200.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A suction stylet assembly for endotracheal intubation, comprising:
an elongated tubular member having a first end and a second end, the elongated tubular member having one or more apertures proximate to the first end; and
a handle member having a first end, a second end, and a side that runs between the first end and the second end, the first end of the handle member in communication with the second end of the elongated tubular member, the handle member including:
a first passageway passing through the handle member running from the first end to the second end, the elongated tubular member coaxial with the first passageway;
a hose fitting located at the second end, the hose fitting configured to receive a hose that provides suction in removing bodily fluids; and
an aperture located on the side, the aperture forming a second passageway through the handle member running between the side and the first passageway, the aperture configured to permit a user to selectively block the aperture with a digit of the user, thereby selectively controlling suction provided between the hose fitting of the handle member and the first end of the elongated tubular member.

2. The assembly of claim 1, wherein the hose fitting is a Christmas tree hose fitting.

3. The assembly of claim 1, wherein at least one of the one or more apertures of the elongated tubular member is a murphy eye.

4. The assembly of claim 3, wherein the murphy eye of the elongated tubular member is concentric with a murphy eye of an endotracheal tube while the elongated tubular member is sheathed by the endotracheal tube.

5. The assembly of claim 1, the handle member further comprising a sheath portion located at the second end, the sheath portion forming a female connector configured to receive a connector of an endotracheal tube.

6. The assembly of claim 1, wherein the first end of the handle member has a tapered portion.

7. The assembly of claim 6, wherein the tapered portion of the first end of the handle member forms a male connector configured to receive a connector of an endotracheal tube.

8. The assembly of claim 1, wherein the elongated tubular member is a malleable metal tube.

9. The assembly of claim 1, wherein the elongated tubular member provides structural support for an endotracheal tube during intubation of a patient.

10. The assembly of claim 1, wherein the elongated tubular member is coated with a soft synthetic material.

11. The assembly of claim 10, wherein the soft synthetic material permits the elongated tubular member to slide against the endotracheal tube.

12. A method of using a suction stylet assembly for endotracheal intubation, the method comprising:
inserting an elongated tubular member of the suction stylet assembly into an endotracheal tube, the elongated tubular member having a first end and a second end, the endotracheal tube having a first end and a second end, the endotracheal tube having a connector located at a second end of the endotracheal tube;
positioning a handle member of the suction stylet assembly in contact with the connector, the handle member having a first end and a second end, the first end of the handle member in communication with the second end of the elongated tubular member, the handle having a hose fitting located at the second end of the handle member;
connecting suction tubing to the hose fitting, the suction tubing providing suction for removing unwanted bodily fluids;
inserting the first end of the elongated tubular member and the first end of the endotracheal tube into a trachea of a patient;
selectively blocking an aperture on the handle member to selectively provide suction to the first end of the elongated tubular member provided by the connected suction tubing; and
removing the suction stylet assembly from the endotracheal tube.

13. The method of claim 12, wherein the aperture is located on a side of the handle member located between the first end and the second end of the handle member.

14. The method of claim 12, wherein selectively blocking the aperture on the handle member is performed with a digit of a user.

15. The method of claim 12, wherein blocking the aperture on the handle member provides suction that draws unwanted bodily fluids located proximate to the first end of the elongated tubular member, through the elongated tubular member and the handle member, and to the connected suction tubing.

16. The method of claim 12, wherein not blocking the aperture on the handle member draws ambient air through the aperture of the handle to the connected suction tubing.

17. The method of claim 12, further comprising:
bending the elongated tubular member and the endotracheal tube into a configuration optimal for intubation, the elongated tubular member maintaining the bent configuration for the endotracheal tube.

18. The method of claim 12, further comprising:
Aligning a murphy hole located proximate to the first end of the elongated tubular member with a murphy hole of the endotracheal tube.

19. The method of claim 12, wherein positioning the handle member in contact with the connector further comprises inserting the connector into a sheath portion located at the first end of the handle member.

20. The method of claim 12, wherein positioning the handle member in contact with the connector further comprises inserting a portion of the first end of the handle member into the connector, wherein the first end of the handle member is tapered.
